(19) United States
(12) Patent Application Publication
(10) Pub. No.: US 2016/0252519 A1
(43) Pub. Date: Sep. 1, 2016

(54) IN-VITRO METHOD FOR DETERMINING FATE OF POLYPEPTIDE VARIANT

(71) Applicant: DR. REDDY'S LABORATORIES LIMITED, Hyderabad (IN)

(72) Inventors: Rajesh MEDISETTY, Hyderabad (IN); Lella RAVI KUMAR, ChiralaMandal (IN); Sridevi KHAMBHAMPATY, Hyderabad (IN)

(21) Appl. No.: 15/030,237

(22) PCT Filed: Oct. 15, 2014
(86) PCT No.: PCT/IB2014/065329

§ 371 Date: Apr. 18, 2016

(30) Foreign Application Priority Data
Oct. 18, 2013 (IN) 4702/CHE/2013

(51) Int. Cl. G01N 33/68

(52) U.S. Cl. CPC G01N 33/6854; G01N 2800/52

(57) ABSTRACT

The present invention discloses an in-vitro method for determining the fate of protein variants upon administration to a subject. Also provided is a means for establishing comparability between two or more distinct therapeutically effective preparations of the same therapeutic protein.

### RELATED APPLICATION

This application is related to and takes priority from Indian Provisional Application 4702/CHE/2013 filed 18 Oct., 2013 and is herein incorporated in its entirety.

### BACKGROUND

The invention describes a method for characterization of the fate of protein variants when administered into a subject, wherein the method is performed in-vitro, but is a predictor of an in-vivo outcome. The disclosed invention could be additionally used to determine in-vivo comparability between two or more distinct preparations of the said therapeutic agent.

Recombinant immunoglobulins have found immense applications in the field of protein based therapeutics. However, production of recombinant immunoglobulins, at an industrial scale, results in formation of numerous immunoglobulin isoforms and variants. These variants include immunoglobulin molecules bearing varied charge, conformation, or glycosylation patterns.

Numerous intrinsic and extrinsic factors are responsible for generation of these variants, including post-translational modification, cell culture conditions, storage buffer conditions etc.

The significance of analyzing these immunoglobulin variants is the premise that these variants may behave differently in-vivo, and therefore influence pharmacokinetic and pharmacodynamic properties of a therapeutic immunoglobulin preparation. For instance, immunoglobulin preparations rich in high mannose glyco-variants are known to clear faster from the serum when compared to other glyco-variants (Goetze et al., 2011). Similarly, afucosylated glyco-variants of immunoglobulins have been associated with enhanced ADCC activity and enhanced affinity to Fc-receptor (Junttilla et al., 2010). Even though no overall biological effects have been ascribed to charge variants, it is nevertheless necessary to understand and assess any potential effects of these variants.

Though the variants of a protein's in-vivo effect are ideally tested in an in vivo setting, due to the inherent constrains associated with an in-vivo assay, it is often necessary to design an in-vitro assays that “mimic” in-vivo outcomes.

The principle object of the present invention is to describe an in-vitro method to determine the fate of immunoglobulin variants in a human subject. A further object of the present invention is to describe a means of establishing biological equivalence between two distinct preparations of the same therapeutic agent.

### SUMMARY

The present invention discloses a method for determining the fate of polypeptide variants in a physiological matrix, wherein the method is performed in an in-vitro setting. The method can, in addition, predict the in-vivo equivalence between two or more distinct therapeutic preparations of the same therapeutic agent.

### BRIEF DESCRIPTION OF DRAWINGS

FIG. 1: Depicts proportions of various charge variants present in two antibody preparations (Ab1 and Ab2) prior to incubation with immunoglobulin depleted serum.

FIG. 2: Depicts proportions of various charge variants present in two distinct antibody preparations (Ab1 and Ab2) of the same therapeutic agent after incubation with immunoglobulin depleted serum.

FIG. 3: Depicts the fate of acidic, basic and main variant of an antibody preparation in time.

### DETAILED DESCRIPTION

Various embodiments of the disclosed invention provide a method for determining the fate of immunoglobulin variants when administered into a subject, wherein the method is performed in an in-vitro setting. The disclosed method in addition provides a means for establishing in-vivo equivalence of two distinct therapeutic preparations of the same therapeutic agent.

As described, immunoglobulin preparations produced by recombinant means typically have numerous isoforms and variants of the protein, including glyco, charge, and conformational variants. The preferred approach for assessing the fate of these variants is to analyze these variants in an in-vivo setting. However, due to inherent constrains associated with in-vivo testing methods, there is a need to develop in-vitro analytical methods that most proximally mimic a physiological matrix.

Immunoglobulins, in particular IgGs, are a major fraction of the serum protein (up to 15 mg/ml of serum). The charge variants of an immunoglobulin arise due to sialylation, deamidation, cleavage of C-terminal lysine (Lyubarskay et al 2006).

The primary variants include acidic variants (negatively charged) or basic variants (positively charged). For instance, sialylation of the protein renders a net negative charge to the protein. Similarly presence of a C-terminal lysine on the heavy chain of an immunoglobulin can confer a net positive charge to the protein. The extent of the net positive charge can vary based on the number of C-terminal lysine residues present. Three main variants have been identified: namely, “K1”, in which one of the heavy chain C-terminal lysine residues is cleaved, “K2”, in which both heavy chain C-terminal lysine residues are preserved, and “K0”, in which both heavy chain C-terminal lysine residues are cleaved.

While, the presence of these charge variants in a therapeutic protein preparation is well recognized, the fate of these variants, when in contact with a physiological matrix, needs to be better understood.

Since, immunoglobulins constitute a significant proportion of the serum; classically, serum immunoglobulin depletion has been employed to study minor protein fractions present in the serum. Further, immunoglobulin depleted serum has been utilized for culturing sensitive hybridomas, so that non-specific immunoglobulin mediated effects could be obviated. However, serum immunoglobulin depletion has not been employed to study fate of polypeptide variants, in particular charge variants, present in a therapeutic polypeptide preparation, neither has the method been used to establish bioequivalence between two or more therapeutic preparations of the same therapeutic agent.

In one embodiment, the claimed invention discloses an in-vitro method for determining the fate of polypeptide variants when a polypeptide preparation is administered to a subject, comprising;

a. Isolating serum or plasma from a subject.

b. Depleting immunoglobulin from the said serum or plasma,

c. Spiking the immunoglobulin depleted serum or plasma with the therapeutically effective polypeptide preparation

d. Incubating the spiked serum or plasma of step c for a first period of time

e. Isolating the polypeptide and variants thereof from the spiked serum or plasma of step d, and

f. Quantifying the polypeptide and polypeptide variants isolated in step e.

In a further embodiment, the polypeptide preparation is an immunoglobulin preparation.

In another embodiment, the claimed invention discloses an in vitro method for demonstration of clinical significance of a polypeptide variant, comprising;

g. Isolating serum or plasma from a subject.

h. Depleting immunoglobulin from the said serum or plasma,

i. Spiking the immunoglobulin depleted serum or plasma with the therapeutically effective polypeptide preparation

j. Incubating the spiked serum or plasma of step c for a first period of time

k. Isolating the polypeptide and variants thereof from the spiked serum or plasma of step d, and

l. Quantifying the polypeptide and polypeptide variants isolated in step e.

In a further embodiment, the polypeptide preparation is an immunoglobulin preparation, and the polypeptide variants are immunoglobulin variants.

In yet another embodiment, the said immunoglobulin variants are charge variants.

In another embodiment, the invention discloses an in-vitro method for establishing in-vivo comparability of polypeptide variants between two or more preparations of a therapeutically effective polypeptide, comprising:

a. Isolating serum or plasma from a subject.

b. Depleting immunoglobulin from the said serum or plasma,

c. Spiking the immunoglobulin depleted serum or plasma separately with the therapeutically effective polypeptide preparations

d. Incubating each of the spiked serum or plasma of step c for a first period of time under identical conditions

e. Isolating the polypeptide and variants thereof from the respective spiked serum or plasma of step d, and

f. Quantifying the polypeptide and polypeptide variants isolated in step e.

g. Comparing the proportion of the polypeptide and polypeptide variants of step e, to demonstrate comparability between the preparations of the therapeutically effective polypeptide.

In a further embodiment, the polypeptide preparations are immunoglobulin preparation and polypeptide variants are immunoglobulin variants.

A number of statistical methods are known in the art to establish comparability between two or more test preparations, such as standard deviation and Student's T-test etc.

A number of techniques and commercial kits are available for serum immunoglobulin depletion as well as for isolation of Ig G from a immunoglobulin G spiked serum or plasma, such as Qproteome Albumin/IgG Depletion Kit, ProteoPrep® IgG depletion kit etc. In absence of commercially available kits, immunoglobulins could be depleted from the serum or plasma by incubating the serum with a protein A or protein G affinity matrix for a suitable amount of time and utilizing the flow-through as the immunoglobulin depleted serum. To ensure effective depletion of immunoglobulin from the serum or plasma, the serum or plasma could be repeatedly incubated with an immunoglobulin affinity matrix.

In another embodiment, the amount of polypeptide preparation for spiking is equivalent to or greater than the serum polypeptide concentration in the subject in whom the polypeptide preparation is administered. The serum polypeptide concentration of a preparation can vary and is dependent on the amount of the preparation administered into a subject. For instance, if administration of 200 mg/m² of a polypeptide preparation leads to a serum polypeptide concentration of 100 μg/ml, then the immunoglobulin depleted serum is to be spiked with at least about 100 μg/ml of the preparation or more.

In a preferred embodiment, the polypeptide or the variant/s thereof are isolated from the immunoglobulin depleted serum by means of a matrix that bears substantial affinity for the said polypeptide or variant/s thereof.

In another embodiment, the various isoforms obtained are quantified physiochemically or biologically. Physiochemical quantification may encompass, without any limitation, methods such as analytical HPLC, isoelectric focusing etc. that disclose physiochemical parameters of a protein. Similarly, biological quantification may include, without any limitation, methods such as antibody dependent cytotoxicity, complement mediated cytotoxicity, in-vitro or in-vivo proliferation assays etc. that disclose biological implications of the polypeptide preparation.

In another embodiment, the said polypeptide preparation is a therapeutically effective polypeptide preparation, wherein the preparation contains one or more variants of the polypeptide.

In another embodiment, the immunoglobulin preparation is a therapeutically effective preparation, wherein the preparation contains one or more variants of the said immunoglobulin. In a particular embodiment, the immunoglobulin preparation is any isotype of IgG.

### EXAMPLE 1

Fate of basic charge variants of two therapeutic antibody preparations of the same therapeutic agent, differing in composition of their charge variants from each other, were compared by incubating these preparations in immunoglobulin depleted serum. The individual antibody preparations were incubated with immunoglobulin depleted serum, followed by isolation of the antibody preparation from the initially spiked serum and quantification of the charge variants of the antibody in both samples.

### Isolating and Depleting IgG From Serum or Plasma

Human serum was freshly prepared from blood, and about 2.3 ml of the serum was incubated with about 1 ml of Protein A/G beads (Cat #89958, Thermo scientific) for about one and half hours at room temperature. At the end of the incubation, the beads were allowed to settle and the harvested supernatant was and again incubated with about 1 ml of Protein A/G beads 9 Cat #89958, Thermo Scientific) for another one and a half hour. At the end of the second incubation the serum supernatant was again harvested. This was deemed to be the IgG depleted serum. Similar method could be followed if IgG were to be depleted from the plasma.

### Spiking the IgG Depleted Serum or Plasma With the Polypeptide Preparation and Incubation

The IgG depleted serum was split into three aliquots, and out of these three, in two separate IgG depleted serum aliquots, about 1 mg of a first test therapeutic antibody preparation (Ab1), and second test therapeutic antibody preparation (Ab2), were added respectively. The amount 1 mg was chosen based on the expected serum concentration of the antibody, if it were administered into a human subject.

Once spiked, the samples were incubated at physiological temperature (about 37° C.) for different periods of time. No antibody was added to the control (C) vial.

### Isolation of the Test Polypeptide

At the end of the incubation all samples Ab1, Ab2 and C were, again incubated with about 0.2 ml of Protein A/G (Cat #89954, Thermo scientific) for 1.5 hrs at RT. At the end of the incubation Protein A/G matrix was washed at least four times with phosphate buffered saline. The antibody bound to the protein A/G matrix was eluted at low pH by using 20 mM Phosphoric acid (pH 1.9-2.3) followed by instant neutralization using 0.2 M disodium hydrogen phosphate (pH 8.9-9.5).

Complete depletion of serum immunoglobulin could be confirmed at this stage, since from the control sample (C) no antibody could be eluted upon incubation with protein A/G matrix, while antibody could be isolated from the spiked serum sample (Table 1)—Represents the amount of antibody obtained either from the control samples or one of the spiked IgG depleted serum samples, following the isolation step of the test antibody preparation from the IgG depleted serum. From the control samples, almost no antibody could be isolated indicating near complete depletion of serum IgG.

TABLE 1
Representation of the amount of obtained antibody.

| | Average absorbance | Protein conc. |
|---|---|---|
| Sample ID | 280 nm | (mg/ml) |
| Control | 0.174 | 0.107 |
| Ab1 | 16.542 | 10.211 |

### Quantification of the Isolated IgG

The eluted antibody from the serum was concentrated in a 30 k Amicon Ultra (0.5 ml) column. The final concentrate was quantified for charge variant distribution by cation exchange chromatography.

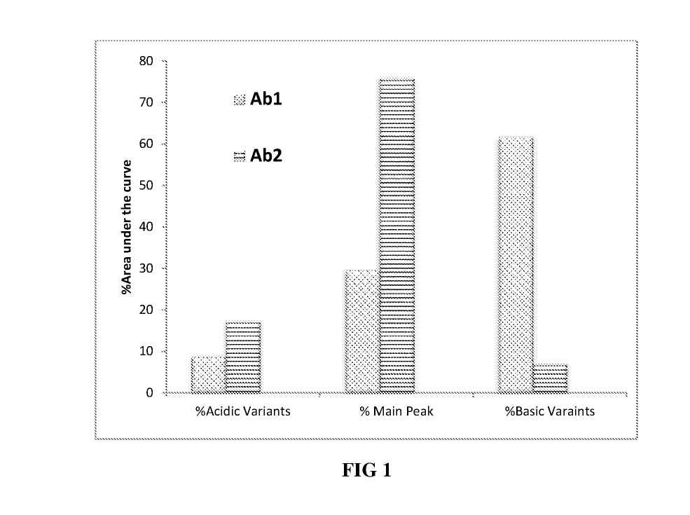

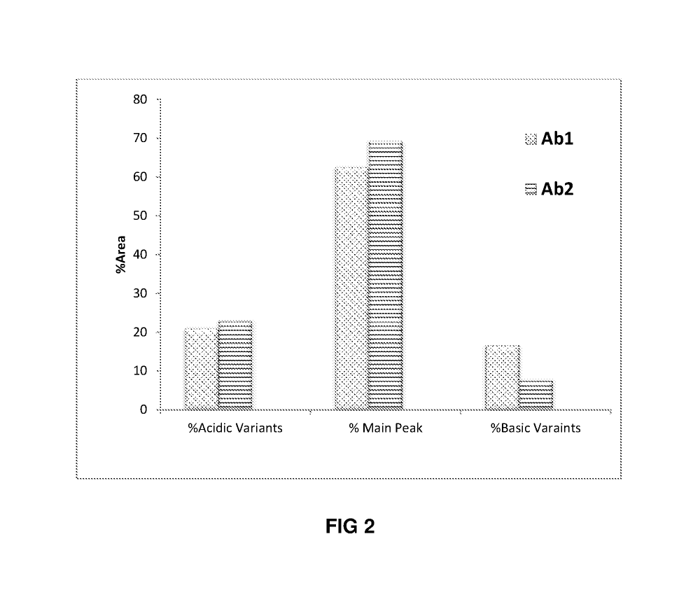

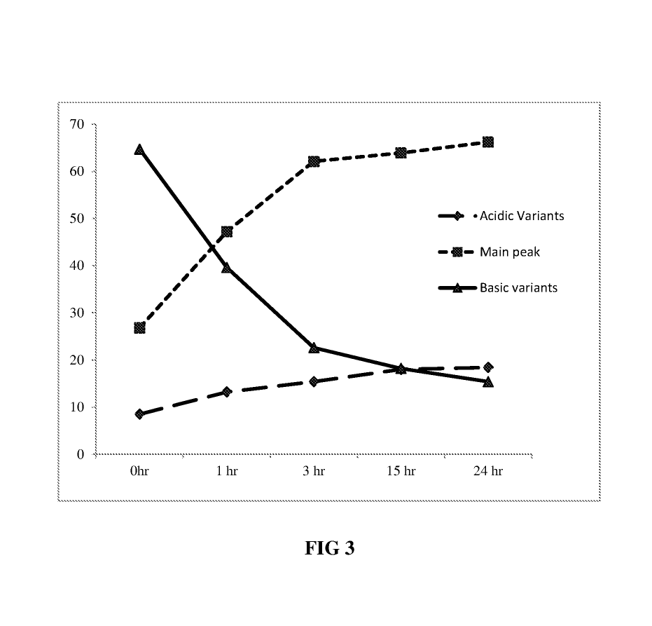

Quantification of the charge variants of Ab1 and Ab2 demonstrated that prior to incubation of the immunoglobulin depleted serum Ab1 contained about 9% acidic variant and about 61% basic variants and the main peak consisted of only about 30% (FIG. 1). Interestingly enough, post incubation of the antibody preparations with immunoglobulin depleted serum contained equivalent proportions of acidic, basic or neutral variants of the protein (FIG. 2). Finally, it can be observed that, upon incubation of the antibody preparation with the immunoglobulin depleted serum, within three hours about 70% of the basic variants are cleared, and this decline is associated with equivalent increase in the proportion of the main variant (FIG. 3).

We claim:

1. A Method of determining polypeptide variants of a polypeptide preparation administered to a subject, comprising;
a) isolating serum or plasma from a subject
b) depleting immunoglobulin from the said serum or plasma,
c) spiking the immunoglobulin depleted serum or plasma with the therapeutically effective polypeptide preparation
d) incubating the spiked serum or plasma of step c for a period of time
e) isolating the polypeptide and variants thereof from the spiked serum or plasma of step d, and
f) quantifying the polypeptide and polypeptide variants isolated in step e.

2). The polypeptide preparation as in claim 1, is a therapeutic polypeptide preparation.

3). The therapeutic polypeptide preparation as in claim 2, includes immunoglobulin and non-immunoglobulin preparation.

4). The polypeptide variants as in claim 1, are selected from the variants group consisting of charge variants and glycovariants

5). An in-vitro method for demonstration of clinical significance of a polypeptide variant of administered polypeptide variants, comprising;
a) Isolating serum or plasma from a subject
b) depleting immunoglobulin from the said serum or plasma
c) spiking the immunoglobulin depleted serum or plasma with the therapeutically effective polypeptide preparation
d) incubating the spiked serum or plasma of step c for a period of time
e) isolating the polypeptide and variants thereof from the spiked serum or plasma of step d, and
f) quantifying the polypeptide and polypeptide variants isolated in step e.
